(19) United States
(12) Patent Application Publication
(10) Pub. No.: US 2017/0303381 A1
(43) Pub. Date: Oct. 19, 2017

(54) PLASMA DEVICE WITH A REPLACEABLE (PLUG-IN) DISCHARGE TUBE

(71) Applicant: Shou Guo WANG, Beijing (CN)

(72) Inventor: Shou Guo WANG, Beijing (CN)

(73) Assignee: Applied Plasma, LLC, Omaha, NE (US)

(21) Appl. No.: 15/111,094

(22) PCT Filed: Jan. 12, 2015
(86) PCT No.: PCT/CN2015/000023

§ 371 Date: Jul. 12, 2016

(51) Int. Cl. H05H 1/46; A61N 1/44

(52) U.S. Cl. CPC H05H 1/46; A61N 1/44; H05H 2245/122; H05H 2001/466

(57) ABSTRACT

This is a pluggable plasma discharge tube device having a replaceable discharge tube and a hand-held shell into which the replaceable discharge tube is plugged. There is a single electrode inside of the tube and no other electrodes outside. This electrode is connected to an output of a power supply and another output of the power supply is connected to a ground wire of its circuit. The input of the power supply is a 12V or lower, DC (direct current) source, or a battery. The plasma is generated via a contact-tube outside discharge, or a plasma jet from the tube, that uses working inert gas. The plasma discharge tube will produce atmospheric pressure, cold quasi-glow plasma, which can be used for sensitive surface disinfection, sterilization, as well as facial skin rejuvenation, treatment of skin tissue infections and destruction of cancer cells.

### TECHNICAL FIELD

This invention involves a plasma device with a replaceable discharge tube. Specifically it is about a plasma discharge tube, which is connected (plugged in) to a hand-held shell. It uses a single electrode to generate quasi-glow cold plasma, which can be used for disinfection and sterilization on sensitive surfaces, facial skin rejuvenation, treatment of skin tissue infections and destruction of cancer cells.

### BACKGROUND

It is generally proven that low temperature cold plasma can rapidly kill germs, viruses and cancer cells. The cold plasma can also be used for facial skin health as well, since the cold plasma contains large quantities of energetic materials such as electrons, ions, and active radicals, which can destroy various harmful bacteria.

The plasma used to kill germs and viruses on the skin requires a sustainable temperature, and in addition, the plasma is required to be glow discharge or quasi-glow, so as not to burn the skin.

Therefore, it is necessary to have low-temperature, cold plasma when applying to the skin. Its discharge intensity should be controllable and uniform, and have a quasi-glow discharge.

Traditionally, the normal capacitive coupling discharge used two electrodes to produce cold plasma between two electrodes. This plasma could be carried out to forma plasma beam by using a gas flow; example patent [public patent ZL200820180894.7], describes this atmosphere pressure plasma stream device. It is a typical dielectric barrier discharge using a capacitive coupling discharge method. A high voltage electrode is inside of an insulation tube, and a ground electrode is outside of the tube. The direction of plasma stream is perpendicular to the direction of the ionized field between the two electrodes. This type of discharge can easily create arcs between the inner high voltage electrode and the skin, while the skin is near to the tube nozzle. Such kind of plasma requires a certain gap to avoid arcs between the skin and tube nozzle. Therefore, it has relatively low energy efficiency. In addition, the capacitive coupling discharge device contains relatively complex manufacturing processes.

Another known method to produce cold plasma is inductive coupling discharge, that uses a solenoid structure with two electrodes connected separately to two output terminals of a power supply. This device [patent KR101260893] has its plasma generated from a spiral tube with coil electrodes. The electrodes are wrapped around the plasma generating tube through the inductively coupled electric field. This kind of discharge cannot be hand-held, and the electrode manufacturing process is difficult.

In patent CN101848595A, the plasma was produced using a radio-frequency (RF) power supply. The device includes a metal RF electrode and a ground electrode. Plasma was produced using the inert gas as a working gas, such as helium. The skin cannot contact the RF electrode during the discharging process. In addition, RF discharges may produce a radiation field, which may bring electromagnetic interference to surrounding electric appliances.

In patent GB2508176A, the plasma generator tip is connected to a RF power supply. This device produces an RF point discharge by directly ionizing the air. This type of discharge cannot be used on the skin and can only be used for cleaning the air.

Currently no pluggable (replaceable tip) design is found in those cold plasma devices. When the plasma is discharged on skin, the part that touches the skin should be replaced for each new patient to avoid cross-contamination.

When interposition plasma is used to treat the body tissue, the gas jetted from the plasma nozzle should be extracted out of the body. That requires the design of the channel of gas extraction. Currently no design of concentric casings suction device has been found.

As seen in past plasma devices, radio frequency and high frequency electric knives are used to cut the skin tissue. They are arc discharge plasmas. These kinds of plasmas are strong enough, though they are not suitable for general skin infection or for killing fungi. This type of device is not suitable for stimulating the activation of tissue cells for the purpose of skin rejuvenation.

In past plasma generation devices, the AC power is usually used. The general power supply voltage is 220V or 110V, power supply is directly turned from AC to DC, forming pulse signal through the high frequency-driven switch tube to drive high voltage transformer to produce high frequency and high voltage output. Using these power supply will cause sense of fear while discharging on the human skin, even if short-circuit protection, overcurrent protection and overvoltage protection are applied. By far no devices of plasma power connection with the low voltage DC power adaptor or battery have been found yet, and no plasma devices with power supply from the USB connection with computer are found for the disinfection of skin surface.

### CONTENTS OF INVENTION

In order to overcome the various shortcomings in the cold plasma found in the devices as described above; this invention provides a plasma generating device with an insulated hand-held housing and a single electrode. In this single-electrode discharge unit, the difficulties in insulation are eliminated compared to devices that have two electrodes, which form a capacitive coupling discharge. This plasma invention also features a simple design and lower production costs.

To avoid cross contamination that might occur when the plasma discharge unit contacts with the skin, this invention has an easy replaceable, plug-in design.

This invention is used for the purpose of disinfection and sterilization of human skin, treatment of skin tissue infections and to kill cancer cells.

In order to better ensure safety when the plasma device discharges on the human body, the plasma generator requires a low-voltage DC power supply. Specifically, it can produce a plasma discharge when connected to the output terminal of a 12V power adaptor, a 5V battery via a USB interface, or powered by a computer USB interface.

The purpose of this power supply design is to isolate it from the common and dangerous 110V/220V source. This plasma power guarantees the safe use of the plasma devices via the connection design of the DC low voltage isolation.

The power supply for this plasma device has a single-electrode output power. The other output terminal is connected to its own ground (as shown in FIG. 2), or may be in suspension (is disconnected). Its output wattage can be controlled with a power control dial or adjusted with a remote digital switch.

The plasma power supply is a unipolar output source that has a voltage range of 4 to 25 kV and a frequency range of 1 to 500 kHz. The power output is 1 to 100 W.

The discharge of the plasma discharge tube is as follows: a metal electrode rod is inserted into an insulating tube with a closed end, with a portion of the metal rod exposed out of the insulating tube. The insulating material of the tube can be glass or ceramic. To accommodate the curved surface processing, the tip of the closed end of the plasma discharge tube may be in different shapes (FIG. 4).

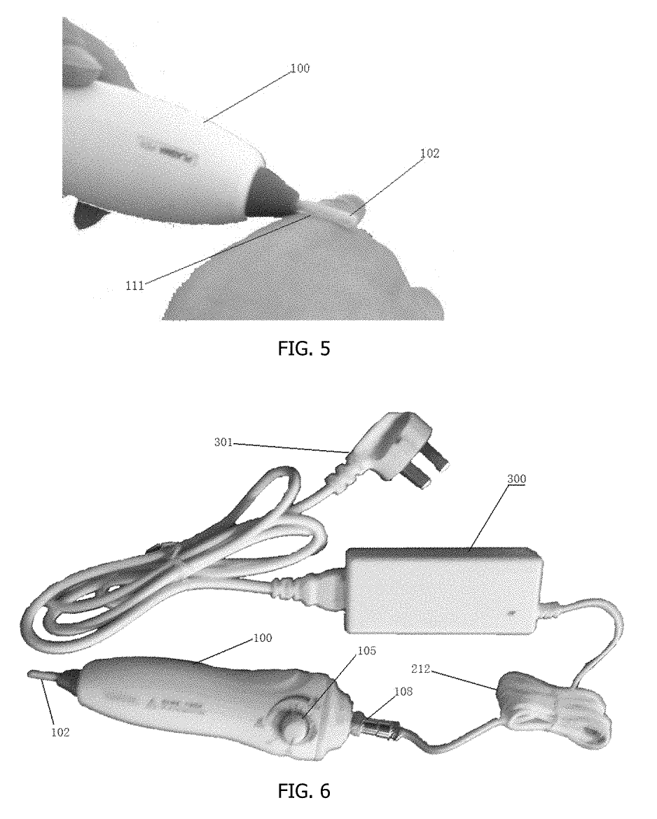

In this invention, the discharge of the plasma discharge tube refers to: when the human body skin is close to the discharge tube within 2 mm or skin contacts the surface of the discharge tube, the space (air) between the skin and tube forms a quasi-polarized plasma charge breakdown (as shown in FIG. 5). If the distance between the skin and discharge tube exceeds 2 mm, plasma is no longer produced.

The plasma discharge tube described in this invention features an exterior discharge mode. This tube is assembled as follows: a metal electrode rod is inserted into an insulating tube with a closed end, a portion of the metal rod exposed outside of the tube. Between the metal electrode and the inner wall of the insulation tube is conductive powder; which may be aluminum, silver, or graphite. The opening of the insulating tube is sealed with sealing gum, which may be conductive silicone.

The plasma discharge tube is connected to the hand-held housing via plugging. The metal electrode rod of plasma discharge tube passing through the rubber location sleeve is plugged into the metal female hole sleeve which is fastened on a plastic bracket inside the shell. The metal sleeve is connected with an output of the power supply and the closed end of the insulating tube is exposed outside of the hand-held shell.

This present invention is a plasma discharge tube, and features: a hand-held, insulated housing equipped with a power supply, a fixed support bracket, connecting wires, rubber material positioning sleeve and metal female hole jack. On the shell body is a power control dial and power input connectors. The advantage of this device is small, lightweight and easy to use inside the hand-held shell.

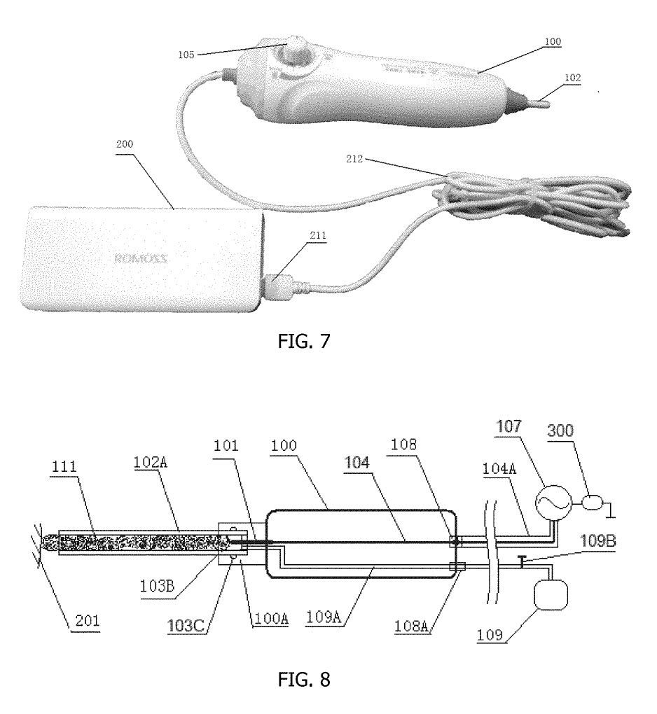

This present invention is a plasma discharge tube, featuring an exterior discharge design. A connection plug is used to connect the power unit inside the shell body with the external power adapter that has a DC output voltage less than 12V (FIG. 6), or connected to a 5V external battery via its USB interface (FIG. 7).

In this invention, the plasma discharge tube uses an inert gas supply featuring a medical plastic tube with both ends open. The plastic tube is connected to plastic connector (a stomatal opening) of the hand-held shell via a plugging. In the plastic connector (stomatal opening) there is an elastic seal. The plastic tube is inserted into the plastic connector (a stomatal opening) and connected through to intake channel of the shell body and to gas supply.

This present invention, plasma discharge tube with the gas supply described herein, features a metal electrode in the air-hole opening (stomatal opening) of the hand-held shell. The end of this electrode is placed inside of the air channel of the shell body and also can be wrapped by the insulating tube. This electrode is connected to an output of plasma power outside of the hand-held shell body through the electrode connector on the hand-held shell body. The other output of the power is connected with the ground wire of its circuit.

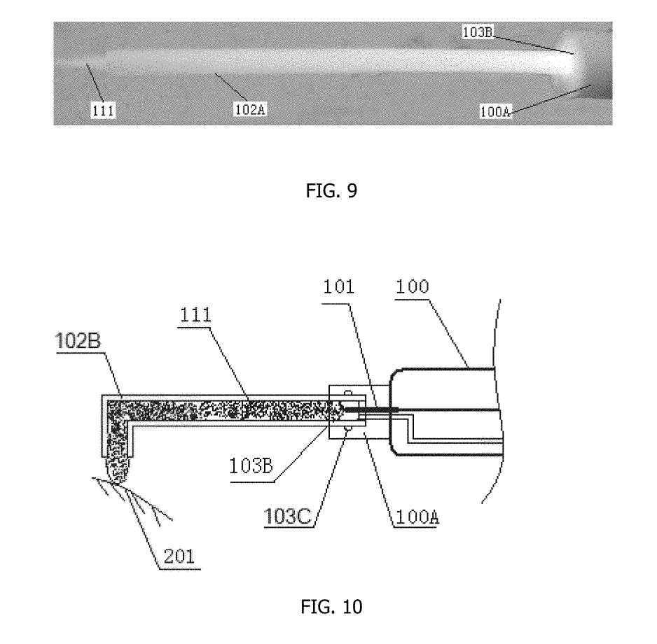

In this invention, the discharge of the plasma discharge tube with an inert gas used refers to: This inert gas flows through the end of the electrode in the shell and is ionized to form a quasi-glow plasma jet stream. This plasma jet stream travels through the plastic tube and is sprayed out of the tube opening (as shown in FIG. 9). The intensity of the plasma jet can be adjusted by the power wattage dial and by a gas volume switch.

In this invention, when the discharge of the plasma discharge tube with an inert gas is used, there should be some space between the nozzle of the plastic tube and the electrode end. The plastic tube is required to be at least 60 mm in length. The length limit is set to avoid an arch discharge when the tube nozzle gets close to the skin.

In this invention, when the discharge of the plasma discharge tube with an inert gas is used, anther concentric outer plastic tube can be added outside of the plastic tube with some gap between the tubes and with outer tube 2 to 20 mm longer than the inner tube. The outer plastic tube is also plugged into the air-hole opening (stomatal opening) of the shell body and connects through to the intake tube in the shell and suction pump out of the shell. The gas sprayed out of the plasma discharge tube is then extracted by the suction pump from the intake channel between the inner and outer tube.

In this invention, the inner discharge of the plasma discharge tube with an inert gas is used. The hand-held shell and the plasma power are connected via cable. The hand-held shell and the gas supply are connected via a gas tube (as shown in FIG. 8).

This present invention uses argon, helium or a mixture of both gases as its gas supply.

### DIAGRAM DESCRIPTIONS

This present invention is further described in the attached diagrams.

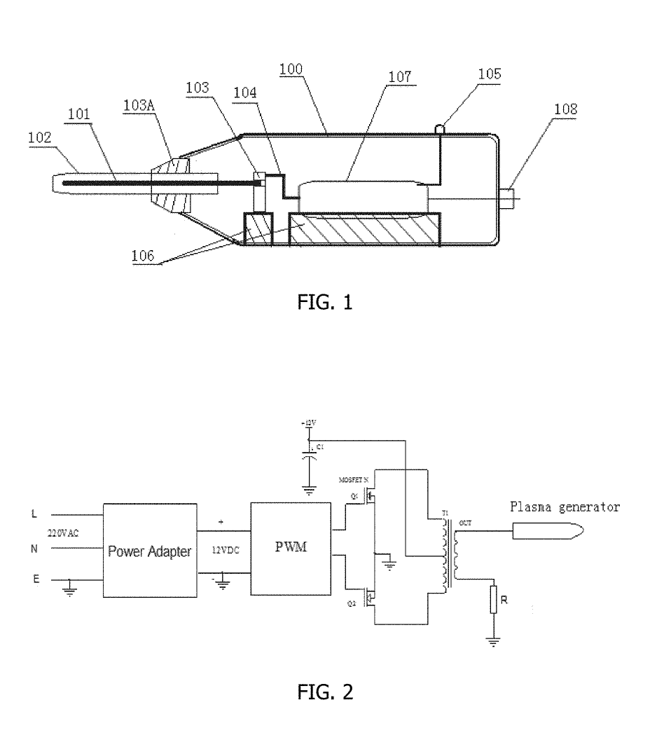

FIG. 1 shows a cross-sectional view of the tube outside discharge structure of the plasma discharge tube. (Implementation Example 1)

FIG. 2 shows the working schematics of the technical implementation.

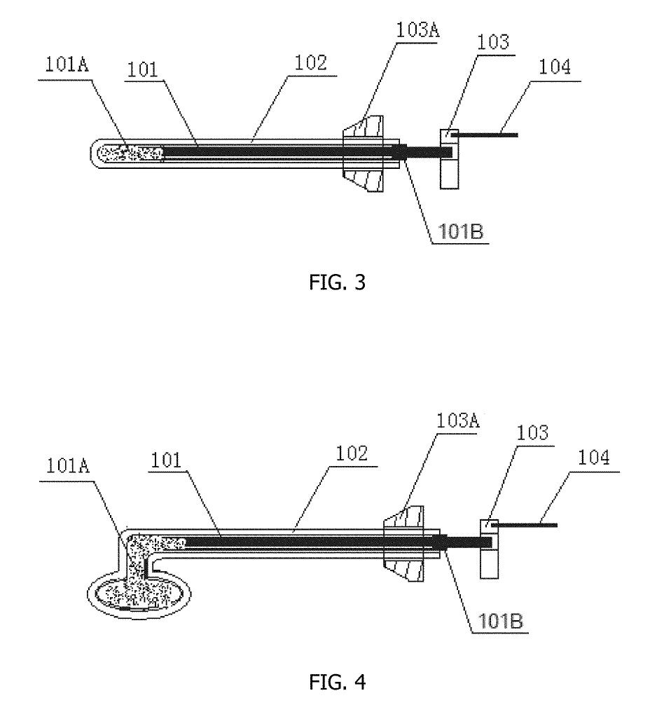

FIG. 3 is a cross-sectional view of the plugging connection schematics in the plasma discharge tube. (Implementation Example 1)

FIG. 4 shows an example of another type of end shape used in the plasma discharge tube. (Implementation Example 2)

FIG. 5 is a photo of this present invention device, as it is being discharged on human skin.

FIG. 6 is a photo of this present invention device with the attached power adapter. (Implementation Example 1)

FIG. 7 is a photo of this present invention device with a USB interface connected to a battery pack. (Implementation Example 1)

FIG. 8 is a cross-sectional view of inner discharge of plasma discharge tube with a gas supply. (Implementation Example 3)

FIG. 9 is a photo of the plasma discharge inside tube with gas supply of this invention. (Implementation Example 3)

FIG. 10 is a cross-sectional view of inner discharge structure of the plasma discharge tube having a gas and with another end shape. (Implementation Example 4)

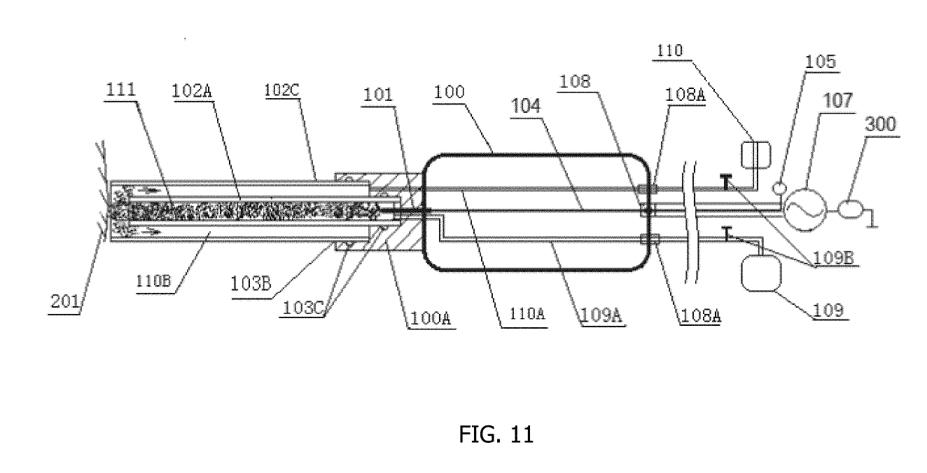

FIG. 11 is a cross-sectional view of the plasma discharge tube having a gas supply and a suction system. (Implementation Example 5)

### REFERENCE LIST OF THE PARTS IN ATTACHED DIAGRAMS

100—hand-held plastic shell housing

100A—plastic connector with center hole, on the housing

101—metal electrode

101A—conductive powder

101B—conductive silicon ring

102—insulation tube (ceramic or glass)

102A—medical plastic tube

102B—medical plastic tube with a bent head

102C—medical plastic outer sleeve

103—metal positioning (location) sleeve

103A—rubber positioning (location) sleeve

103B—circular gas opening

103C—rubber sealing ring

104—wire

104A—insulation wrapped wire

105—power (wattage) control dial

106—insulated positioning bracket

107—single (unipolar) output power supply

108—electrode connection plug

108A—gas tube connector plug

109—gas source

109A—intake tube

109B—gas volume control dial

110—suction pump

110A—exhaust tube

110B—space between two plastic tubes

111—plasma

200—battery

201—skin

211—USB connector

212—twin connecting wire

300—power adapter

301—power plug

### SPECIFIC IMPLEMENTATION METHODS

### Implementation Example 1

As shown in FIGS. 1, 2, 3 and 5 of this present invention, these are the details for Example 1 implementation. A hand-held shell [100] which encloses a plasma power unit [107], plastic positioning bracket [106], metal positioning sleeve [103] and connecting wire [104] is shown. In the shell casing there are such components as a wattage adjustment control dial [105], and a power supply (electrode) connector plug [108]. The integrated components of this device allows for easy operation, and are lightweight and small in size.

As shown in FIGS. 1 and 3, the plasma discharge tube is connected via plugging to the hand-held shell through the positioning sleeve [103A]. The metal electrode [101] goes through the rubber positioning sleeve [103A], fastened on the shell [100], and is inserted into a metal sleeve [103] and connected to the metal sleeve. The metal sleeve [103], made of copper or stainless steel, is placed on an insulation bracket [106] of the shell [100] and connected to an output of power [107]. The closed end of the insulation tube [102] is exposed out of the hand-held shell [100]. The advantages of the plugging connection method between the plasma discharge tube and the shell [100] include precise positioning, convenient plugging, easy replacement after each use.

As shown in FIG. 3, the plasma discharge tube is assembled as follows: A metal electrode is inserted inside an insulation tube [102] which is enclosed on one end. This metal electrode [101] can be made of copper, stainless steel, or tungsten copper alloy. Part of the electrode [101] is exposed outside of the insulation tube [102]. The gap between the electrode [101] and the inner wall of the insulation tube is filled with a conductive powder [101A], which may be aluminum, silver, or graphite. At the open end of the insulation tube [102] is a sealing ring [101B], which is made of conducting silicon. Fabricated this way, the plasma electrode tube does not have any air gaps and avoids unwanted discharge inside the tube.

As shown in FIG. 5, the outside discharge of the plasma discharging tube: when the skin is within 2 mm from the discharge tube or contacts the surface of discharge tube (insulation tube) [102], the air space between the skin and tube is disrupted by a polarized charge on the tube [102] surface. This interaction produces a quasi-glow cold plasma discharge. If the discharge tube is more than 2 mm away from the skin, no plasma is produced. This discharge of the plasma occurs in the air, and no extra operating gas is needed. The intensity of the plasma discharge is regulated by the power control dial [105].

As shown in FIGS. 6 and 7, the transformer output of plasma power supply [107] of this plasma device requires an voltage range between 4 kV and 15 kV, and a frequency range between 1 kHz and 500 kHz, a wattage of the power supply between 0 W and 30 W. The other output of the power supply is connected to its own ground wire [FIG. 2].

The power source [107] of the plasma discharge device requires a low voltage DC power input, under 12V. For example, the power adapter [300] in (FIG. 6) may be used, or a USB port [211] can be connected to a 5V battery [200] in (FIG. 7). This power supply connection of plasma power supply [107] guarantees the safety when the plasma discharge is applied to the skin.

### Implementation Example 2

As shown in FIG. 4, a bent head is used for the plasma discharge tube's end in Example 2 implementation, and that is the only differences between Example 2 implementation and that shown in Example 1. Compared with the shape of straight tube in Example 1, this discharge insulation tube with the closed end elbow-shaped produces plasma on the arched surface of its closed end. It is easy to observe the discharge from the side. All the other structures and functions of this present invention remain the same as Example 1 implementation.

In the embodiments of Example 1 and 2 implementations, one end of the plasma discharging tube is inserted into a metal positioning sleeve [103], which is connected via wire [104] to the output terminal of the power supply [107]. These connections are shown in FIGS. 1, 2, 3 and 4. FIG. 2 shows a diagram of the connection of a single plasma electrode and its power supply. No other peripheral electrodes are needed with the plasma discharging tube.

### Implementation Example 3

As shown in FIG. 8, the plasma discharge tube has a gas supply source and a internal discharge method. The device adopts a medical plastic tube [102A], with both end open, which is plugged into the connector [100A] of the hand-held shell [100]. This connector [100A] has a circular gas opening [103B] that enables a plug-in connection. Inside the circular gas opening [103B] is an elastic sealing ring [103C]. The medical plastic tube [102A] is plugged into the circular gas opening [103B] and connected through to the intake tube [109A] inside the hand-held shell [100]. This connection is used to form a channel through which gas may flow from the gas source [109].

As shown in FIG. 8, there is a single metal electrode [101] inside the opening of the hand-held shell [100]. This metal electrode [101] can be made of copper, stainless steel, or Tungsten copper alloy. The top end of the metal electrode [101] is inside the gas channel of the plastic shell [100]. The exterior of the metal electrode can also be wrapped with a ceramic or quartz tube. This metal electrode [101] is connected via wire [104] to the electrode connector plug [108] that is attached to the hand-held plastic shell [100]. The electrode connector [108] is then connected by an insulated wire [104A] to an output terminal of a high voltage, high frequency power supply [107] that is outside of the shell [100]. The other output terminal of the power supply [107] is connected to a ground in its own circuit. The input end of the power supply [107] is connected to a power adapter [300], which provides 12V (or lower) DC.

As shown in FIGS. 8 and 9, when the inert gas from source [109] reaches the top end of the electrode [101], it is ionized to form a plasma stream (jet) [111], which travels through the medical plastic tube [102A] and sprays out of the tube tip. The strength of the plasma can be controlled by adjusting the output capacity of the power and the gas volume control dial [109B].

As shown in FIG. 8, the nozzle of the plastic tube [102A] must be kept a minimum distance from the end of the electrode [101]. The length of the plastic tube [102A] shall be a minimum of 60 mm. This limit of length avoids an arc discharge of the electrode [101] direct on human body when human skin gets close to the nozzle of plastic tube.

As shown in FIG. 8, the Example 3 implementation features an internal discharge of plasma discharge tube with an inert gas source. The hand-held shell [100] is connected to a power supply [107] via an insulated wrapped wire [104A], and is connected to the inert gas source [109] via the intake tube [109A].

The gas source [109] is inert gas which can be argon, helium or the mixture of the two.

The output of power supply [107] in the Example 3 implementation is required to have a voltage between 4 and 25 kV, a frequency of 1 to 500 kHz and power wattage of 1 to 100 W. The other end of the power supply [107] is connected to its own ground as shown in FIG. 2.

The power supply [107] required in the Example 3 implementation is a 12V (or lower) DC input. For example, the power supply [107] may be connected to a power adapter [300], which has an output voltage of 12V, as shown in FIG. 6. This type of power supply [107] ensures the safety, when discharge is applied on the skin, via low voltage current isolation from the usual energy of a 220V or 110V power source.

### Implementation Example 4

As shown in FIG. 10 is the schematic diagram of the structure of the plasma tube, in Example 4 implementation, in another end shape, discharging inside and with gas supply source. The difference from Example 3 implementation is the elbow-shaped end plastic tube [102B] used. In this case, plasma discharge may be applied are used in the treatment of oral skin infections.

### Implementation Example 5

As shown in FIG. 11, is a cross-sectional view of the Example 5 implementation of this present invention that has an inert gas supply, a suction system and an internal discharge mode. In this diagram, an inner plastic tube [102A] has an outer concentric plastic sleeve [102C]. A space [110B] of 1 mm or less is kept between inner [102A] and outer plastic tube [102C]. The outer plastic sleeve [102C] is 2 to 20 mm longer than the inner plastic tube [102A]. The outer plastic sleeve [102C] is plugged into the plastic connector [100A] of the shell [100] and connects with the exhaust tube [110A] inside handheld shell [100] and suction pump [110] outside handheld shell [100], to form a continuous air/gas flow. The air sprayed from the end of the plastic discharge tube [102A] is then extracted out through the space [110B] between the outer plastic sleeve [102C] and the inner plastic tube [102A] and through the exhaust tube [110A] by the suction pump [110].

As shown in FIG. 11 is Example 5 implementation. Its electrode structure, connections, gas supply and power supply requirements are exactly the same as in Example 3. The connecting inner plastic tube [102A] and the outer plastic sleeve [102C] are both made of medical plastic. The tube and sleeve can be a joint structure as long as there is a proper space between them.

As shown in FIG. 11 is Example 5 implementation. The purpose of the suction system is for interventional plasma therapy on the human body. The plasma gas flowing out of the discharge tube end is then extracted out of human body through the suction pump [110] and the exhaust tube [110A].

The above references and the diagrams provides the descriptions of this invention in detailed implementation examples. However, it is necessary to point out that changes and modifications can be made to the above implementation examples by technical people in this area under the circumstance of not departing from the spirit and scope of this utility model. The changes and modifications must be within the constraints set forth in this patent claim of the utility model.

1-13. (canceled)

14. A plasma generating device comprising:
a housing being hand-held, defining a junction for connection to a discharge tube, having a source of power or a source of power input connector, and having a power output connector electrically connected to the source of power or the source of power input connector; and
a sole electrode within a discharge tube that is releasably attachable to the housing at the junction;
wherein the sole electrode is electrically connected to the power output connector and at least a portion of the discharge tube protrudes outward from the housing;
wherein the source of power has a maximum of 12 volts direct current that is connected to a ground wire or is disconnected, and the discharge tube generates atmospheric cold quasi-glow plasma.

15. The plasma generating device of claim 14, wherein the power output connector comprises a metal sleeve defining a female hole into which the sole electrode is fitted for electrical connectivity.

16. The plasma generating device of claim 15, wherein the junction for connection to a discharge tube comprises a rubber sleeve defining a hole having a pluggable fit to the discharge tube.

17. The plasma generating device of claim 14, wherein the discharge tube has a distal end relative to the housing that is closed and a conductive powder enclosed within the discharge tube surrounding the sole electrode.

18. The plasma generating device of claim 17, wherein the discharge tube generates atmospheric cold quasi-glow plasma when skin of a patient is within 2 mm or less from the exterior surface of the discharge tube.

19. The plasma generating device of claim 14, wherein the discharge tube comprises an insulating material selected from the group consisting of ceramic, glass, or plastic.

20. The plasma generating device of claim 14, wherein the discharge tube has a distal end relative to the housing that is open, and a source of inert gas connected in fluid communication with the discharge tube when releasably connected to the housing; wherein as the inert gas reaches a distal end of the sole electrode, the inert gas is ionized to form a plasma stream that sprays out the open distal end of the discharge tube.

21. The plasma generating device of claim 20, wherein the discharge tube further comprises an outer sleeve concentric therewith and spaced a distance apart therefrom, the outer sleeve being 2 to 20 mm longer than the discharge tube; wherein the outer sleeve is releasably attachable to the junction for receiving the discharge tube for fluid communication with a source of suction, thereby extracting the inert gas from the plasma stream before it exits the outer sleeve.

22. The plasma generating device of claim 1, wherein the source of power is a battery.

23. The plasma generating device of claim 1, wherein the source of power is a power adaptor having a power plug, the power adaptor reducing the voltage to a maximum of 12 volts direct current.

24. The plasma generating device of claim 1, wherein the housing further comprises a power output control device.

25. The plasma generating device of claim 10, wherein the source of power is a unipolar output source having a voltage range of 4 to 25 kV, a frequency range of 1 to 500 kHz, and a power output of 1 to 100 W.

26. A method for treating skin comprising
providing a plasma generating device of claim 1;
turning on the plasma generating device; and
generating atmospheric cold quasi-glow plasma at an area of skin in need of treatment.

27. The method of claim 26, wherein the skin in need of treatment requires disinfecting or sterilizing.

28. The method of claim 26, wherein the skin in need of treatment comprises cancer cells or an infection.

29. The method of claim 26, wherein the discharge tube of the plasma generating device has a distal end relative to the housing that is closed and a conductive powder enclosed within the discharge tube surrounding the sole electrode, and the method further comprises positioning the skin in need of treatment within 2 mm or less from an exterior surface of the discharge tube.

30. The method of claim 26, wherein the discharge tube of the plasma generating device has a distal end relative to the housing that is open, and a source of inert gas connected in fluid communication with the discharge tube when releasably connected to the housing; wherein as the inert gas reaches a distal end of the sole electrode, the inert gas is ionized to form a plasma stream that sprays out the open distal end of the discharge tube; the method further comprising positioning the open distal end of the discharge tube proximate the skin in need of treatment.

31. The method of claim 30, wherein the discharge tube further comprises an outer sleeve concentric therewith and spaced a distance apart therefrom, the outer sleeve being 2 mm to 20 mm longer than the discharge tube; wherein the outer sleeve is releasably attachable to the junction for receiving the discharge tube for fluid communication with a source of suction, thereby extracting the inert gas from the plasma stream before it exits the outer sleeve.
